(19) United States
(12) Patent Application Publication
(10) Pub. No.: US 2019/0321642 A1
(43) Pub. Date: Oct. 24, 2019

(54) IMPLANTABLE SYSTEM

(71) Applicants: UNIVERSITE GRENOBLE ALPES, Saint Martin D'Heres (FR); CENTRE HOSPITALIER UNIVERSITAIRE GRENOBLE ALPES, La Tronche (FR)

(72) Inventors: Philippe CINQUIN, Saint Nazaire Les Eymes (FR); Pascal DEFAYE, Grenoble (FR); Patrick TUVIGNON, Albi (FR)

(21) Appl. No.: 16/467,625

(22) PCT Filed: Dec. 7, 2017
(86) PCT No.: PCT/EP2017/081889

§ 371 Date: Jun. 7, 2019

(30) Foreign Application Priority Data
Dec. 7, 2016 (FR) 1662058

(51) Int. Cl. A61N 1/378; A61N 1/36

(52) U.S. Cl. CPC A61N 1/3785; A61N 1/36007

(57) ABSTRACT

An implantable system comprising a first device, also called a centralisation device, suitable for being implanted in a fixation position inside the patient's body and at least one second device suitable for stimulating an organ of the patient when the second device is implanted, in a stimulation position, in the patient's body. The first device being further configured to command a stimulation of the organ by the second device. The implantable system being characterised in that the organ is a separate organ from the stomach and in that, when the first device is in the fixation position, the first device is accommodated in the patient's stomach and fixed to a stomach wall.

The present invention relates to an implantable system.

A large number of implantable devices are used for monitoring or stimulating certain organs of the human body. For example, cardiac stimulation devices (or “pacemakers”) are implanted in many patients. These devices generally include a power source such as a battery, one or a plurality of sensors for monitoring the behaviour of the organ monitored and/or a stimulation module envisaged to exert an action on the stimulated organ.

However, it is necessary to recharge or replace the batteries of such implantable devices regularly. In particular, in numerous cases, this replacement is carried out by means of a surgical procedure. Such a procedure is relatively expensive and restrictive for the patient since it takes place in a hospital facility operating theatre and anaesthesia is required, as well as a prolonged stay in the hospital facility for the purposes of post-operative monitoring. Furthermore, as for any surgical procedure, there are risks of the patient contracting an infection during the operation.

In other cases, implanted devices of the type mentioned above are externally powered by an energy storage module which is borne by the patient outside their body. For example, some power supply devices may transmit energy via ultrasound waves to the stimulation device, through the patient's skin and rib-cage. However, ultrasound waves pass poorly through bones, and great precision in the placement of the ultrasound source is then required, in cases where the implanted device is situated in front of the rib-cage, so as to provide a satisfactory power supply of the implanted device. Furthermore, such a power supply device outside the patient's body is unsightly.

Some implantable devices may be equipped with wired connectors, enabling an electrical connection or fluid transfer between the implantable device and an external device. In this way, an electrical power supply current or data measured by the sensors of the implanted device are exchanged with the external device. Here again, these connectors emerging through the patient's skin are unsightly, and necessarily involve health risks as well as significant constraints for the patient's day-to-day life.

Therefore, there is a need for an implantable system that is less restrictive for the patient.

For this purpose, there is proposed an implantable system comprising a first device, also called centralisation device, suitable for being implanted in a fixation position inside the patient's body and at least one second device suitable for stimulating an organ of the patient when the second device is implanted, in a stimulation position, in the patient's body, the first device being further configured to command a stimulation of the organ by the second device, the implantable system being characterised in that the organ is a separate organ from the stomach and in that, when the first device is in the fixation position, the first device is accommodated in the patient's stomach and fixed to a stomach wall.

According to the embodiments, the implantable system comprises one or a plurality of the following features, taken in isolation or according to any technically possible combinations:
the first device is configured to supply the second device with power;
the centralisation device includes an acoustic wave emitter and the second device includes a stimulator suitable for stimulating the organ and a converter of acoustic energy into electrical energy, the converter being suitable for receiving the acoustic waves emitted by the centralisation device and for generating in response an electrical power supply current of the stimulator;
the acoustic wave is an ultrasound wave;
the centralisation device includes an electromagnetic wave emitter and the second device includes a stimulator suitable for stimulating the organ and a converter suitable for receiving the electromagnetic waves emitted by the centralisation device and for generating in response an electrical power supply current of the stimulator;
the organ is the patient's heart;
the implantable system includes at least two second devices;
a second device is envisaged to be implanted in the right ventricle and a further second device is envisaged to be implanted in the left ventricle of the patient's heart;
the organ is a nerve of the patient;
the organ is the patient's phrenic nerve;
the organ is the patient's diaphragm;
when the centralisation device is in the fixation position, the centralisation device is accommodated in the upper part of the stomach;
the centralisation device or the second device includes at least one sensor suitable for measuring at least one value of a parameter of the organ and the centralisation device comprises a controller suitable for commanding the stimulation of the organ, by the second device, according to the measured value(s);
the centralisation device comprises a controller and an electrical power supply including a removable electrical energy reserve and a connector suitable for accommodating the electrical energy reserve, the electrical energy reserve being suitable for electrically powering the controller when the electrical energy reserve is connected electrically to the connector in a connection position and preferably being configured to be swallowed by the patient and to move spontaneously to the connection position from a disconnection position wherein the electrical energy reserve is accommodated in the stomach of the patient (P) and is disconnected from the connector;
the centralisation device comprises a controller and an electrical power supply suitable for generating an electrical power supply current of the controller by reacting at least one chemical species present in the patient's body, particularly glucose;
the centralisation device comprises a controller and an electrical power supply suitable for generating an electrical power supply current of the controller by converting mechanical energy into electrical energy.

Features and advantages of the invention will emerge on reading the following description, given merely by way of non-limiting example with reference to the appended drawings, wherein:

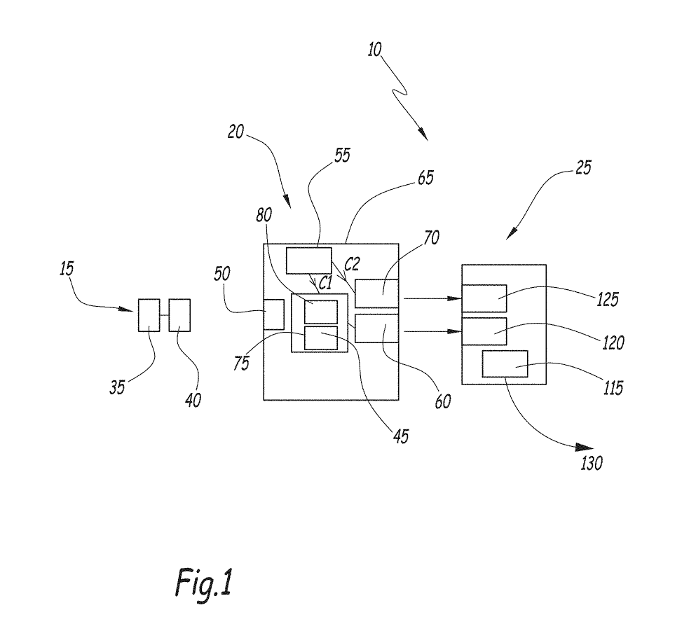

FIG. 1 is a diagram of an example of an implantable system including an electrical power supply,

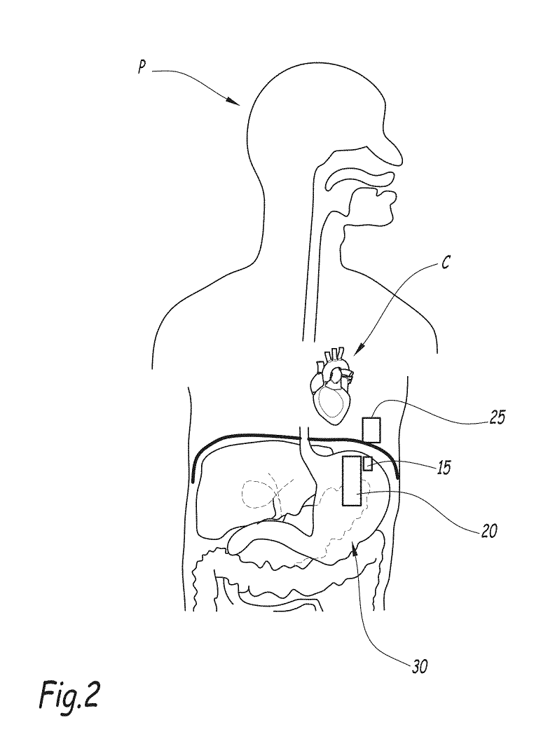

FIG. 2 is a schematic representation of the implantable device in FIG. 1, implanted in a patient's body, and

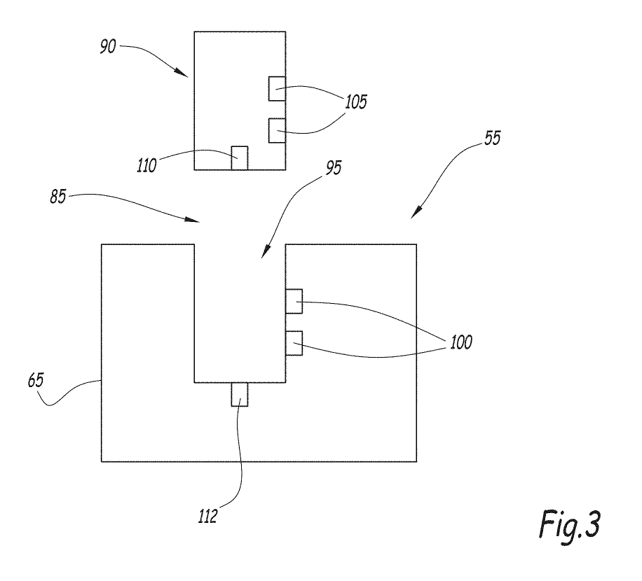

FIG. 3 is a schematic representation of the power supply in figure

A first example of an implantable system 10 is represented in figure

The implantable system 10 includes an anchor 15, a first device 20, also called centralisation device, and at least one second device 25.

It is understood by “implantable system” that at least one element from the list formed by the anchor 15, the first device 20 and the second device 25 is envisaged to be implanted in the human body.

In particular, it is understood by “implantable” that at least one element from the anchor 15, the centralisation device 20 and the second device 25 is envisaged to remain in the body of a patient P for a period strictly greater than one week, preferably greater than one month, preferably greater than or equal to one year.

The implantable system 10 has been represented schematically in FIG. 2 when the implantable system 10 is implanted in the body of the patient P.

According to the example in FIG. 2, the anchor 15, the centralisation device 20 and the second device 25 are each implanted in the body of the patient P.

The anchor 15 is suitable for being fixed in a predetermined position in the stomach 30 of the patient P.

For example, the anchor 15 is configured to be fixed in the upper part of the stomach 30. In particular, the anchor 15 is configured to be fixed in the gastric fundus of the stomach 30. For example, the anchor 15 is envisaged to be fixed as close as possible to the angle of His in the gastric fundus.

Alternatively, the anchor 15 is configured to be fixed in the lower part of the stomach 30.

The anchor 15 is configured to support the centralisation device 20, preferably removably. In particular, the anchor 15 and the centralisation device 20 are configured to be fixed to one another, by a fixation device, and the anchor 15 is configured to hold the centralisation device 20 in a fixation position when the anchor 15 is fixed in the stomach 30.

The anchor 15 includes a head 35 and a first connector 40.

The head 35 is configured to anchor the anchor 15 in the predetermined position. In particular, the head 35 is configured to anchor the anchor 15 to the wall of the stomach 30.

The head 35 is, for example, a gastrointestinal clip configured to grip between two branches of the head 35 a portion of the wall of the stomach 30.

Alternatively, the head 35 is suitable for being sutured using a suture to the wall of the stomach 30.

According to a further alternative embodiment, the head 35 is suitable for being embedded inside the gastric mucosa after the latter has been dissected.

The first connector 40 is configured to fix the centralisation device 20 to the head 35.

The centralisation device 20 is configured to supply the second device 25 with power.

The centralisation device 20 includes a first controller 45, a second connector 50, an electrical power supply 55, a first emitter/receiver 60, a housing 65 and a transmitter 70.

The first controller 45 is a data processing unit. The first controller 45 includes a first memory 75 and a first processor 80.

Alternatively, the first controller 45 is embodied in the form of a dedicated integrated circuit, or programmable logic components.

The first processor 80 is suitable for processing and/or converting data represented as electronic or physical quantities in the first memory 75 into other similar data corresponding to physical data in the first memory 75, in registers or other types of display, transmission or storage devices.

The first processor 80 is further configured to exchange data with the first emitter/receiver 60.

The second connector 50 is configured to cooperate with the first connector 40 to hold the centralisation device 20 in the fixation position.

For example, the second connector 50 is configured to cooperate with the first connector 40 by snap-fitting.

Alternatively, the second connector 50 includes a magnet configured to fix the second connector to the first connector. The magnet is, for example, an electromagnet.

According to a further alternative embodiment, the first connector 40 is configured to be secured to the second connector 50 by screwing.

Alternatively, the first connector 40 includes one or preferably two bayonets complementary with fixation orifices formed in the second connector 50.

Preferably, the second connector 50 is envisaged such that the centralisation device 20 is separable from the anchor 15. In particular, the second connector 50 is configured such that the centralisation device 20 is separable from the anchor 15 when the anchor 15 is fixed in the stomach 30 of the patient P.

The electrical power supply 55 has been represented in FIG. 3.

The electrical power supply 55 is configured to supply the first controller 45 with a first power supply current C1.

The electrical power supply 55 is further configured to supply the transmitter 70 with a second power supply current C2.

The electrical power supply 55 includes a third connector 85 and a first electrical energy reserve 90.

The third connector 85 is configured to receive from the first electrical energy reserve 90 the first power supply current C1 and the second power supply current C2 and to supply the first controller 45 and the transmitter 70 with, respectively, the first power supply current C1 and the second power supply current C2.

The third connector 85 is configured to accommodate the first electrical energy reserve 90. In particular, the third connector 85 delimits a cavity 95 configured to accommodate at least partially the first electrical energy reserve 90 in a connection position.

According to the example in FIG. 3, the cavity 95 emerges outside of the housing 65. In particular, the cavity 95 is configured to enable the insertion of the first electrical energy reserve 90, from outside the housing 65, into the cavity 95.

The third connector 85 further includes two first electrical contacts 100, configured to be connected electrically to the first electrical energy reserve 90 when the first electrical energy reserve 90 is in the connection position. In particular, the two first electrical contacts 100 emerge inside the cavity 95.

The first electrical energy reserve 90 is configured to store electrical energy. In particular, the first electrical energy reserve 90 is configured to be charged with electrical energy outside the body of the patient P and to be discharged when the first electrical energy reserve 90 is in the connection position. For example, the first electrical energy reserve 90 includes a battery. Alternatively, the first electrical energy reserve 90 includes at least one capacitor or a supercapacitor.

The first electrical energy reserve is configured to supply the first controller 45 with the first power supply current C1 when the first electrical energy reserve 90 is in the connection position. Furthermore, the first electrical energy reserve 90 is configured to supply the transmitter 70 with the second power supply current C2 when the first electrical energy reserve 90 is in the connection position.

According to the example in FIG. 3, the first electrical energy reserve 90 includes two second electrical contacts 105 complementary with the first electrical contacts 100.

The first electrical energy reserve 90 may be envisaged to be swallowed by the patient P.

According to an alternative embodiment, the first energy reserve 90 is suitable for being replaced by endoscopy.

In particular, the first electrical energy reserve 90 has a volume strictly less than 6 millilitres (ml).

The first electrical energy reserve 90 further has three dimensions each measured along a respective direction, each direction being perpendicular to the two other directions, and each dimension is strictly less than 5 centimetres (cm).

The first electrical energy reserve 90 is movable between the connection position and a disconnection position. When the first electrical energy reserve 90 is in the disconnection position, the first electrical energy reserve 90 is accommodated in the stomach 30 of the patient P but is not connected electrically to the third connector 85. For example, when the first electrical energy reserve 90 is in the disconnection position, the first electrical energy reserve is fully removed from the cavity 95.

The first electrical energy reserve 90 is configured to move spontaneously from the disconnection position to the connection position. For example, the first electrical energy reserve 90 includes attractors 110.

The attractors 110 are configured to exert on the first electrical energy reserve 90, when the first electrical energy reserve 90 is in the disconnection position, a force tending to move the first electrical energy reserve 90 from the disconnection position to the connection position.

Furthermore, the attractors 110 are configured to hold the first electrical energy reserve 90 in the connection position.

The attractors 110 include, for example, a first magnet suitable for cooperating with a second magnet 112 of the third connector 85. Alternatively, the first magnet is suitable for cooperating with a ferromagnetic portion of the third connector 85. The first magnet and the second magnet 112 are, for example, electromagnets.

The first emitter/receiver 60 is configured to exchange data with the second device 25. The first emitter/receiver 60 thus forms communication means with the second device 25.

The first emitter/receiver 60 is, for example, a radiofrequency communication module. It is understood by “radiofrequency communication module” that the first emitter/receiver 60 is configured to communicate with the second device 25 via a signal including at least one radiofrequency electromagnetic wave. The radiofrequency electromagnetic waves are electromagnetic waves having a frequency between 3 kilohertz and 3 gigahertz.

According to one embodiment, the first emitter/receiver 60 is suitable for exchanging data with the second device 25 according to a Bluetooth Low Energy protocol. The Bluetooth Low Energy protocol is a protocol based on a “Bluetooth special interest group” standard and functioning in the range between 2400 megahertz (MHz) and 2483.5 MHz.

Alternatively, data transmission modes in the 402-405 megahertz (MHz) (Medical Implant Communication Service) or 2360-2390 MHz (Medical Body Area Networks) ranges may be used.

The housing 65 is configured to isolate the first controller 45 from outside the housing 65. For example, the housing 65 delimits a chamber receiving at least the first controller 45, the first emitter/receiver 60 and the transmitter 70.

The transmitter 70 is configured to transmit power to the second device 25.

For example, the transmitter 70 includes an acoustic wave emitter/receive configured to emit an acoustic wave and to route the acoustic wave to the second device 25.

Alternatively, the transmitter 70 is configured to emit an electromagnetic wave. The electromagnetic wave has, for example, a high frequency such as a frequency in the region of 13.56 Megahertz (MHz). For example, the transmitter 70 includes a coil configured to emit the electromagnetic wave.

Alternatively, the transmitter 70 is connected to the second device 25 by an electrical conductor and is configured to transmit an electric current to the second device 25 via a wired link.

The second device 25 is suitable for stimulating an organ C of the patient P.

The organ C is separate from the stomach 30 of the patient P.

The second device 25 is configured to be implanted in the body of the patient P in a stimulation position. The second device 25 is then configured to stimulate the organ C when the second device 25 is in the stimulation position.

When the second device 25 is in the stimulation position, the second device 25 is situated outside the stomach 30.

It is understood by “stimulate” that the second device 25 is suitable for exerting an action on the organ C and triggering in response an action of the organ C. A muscle contraction is an example of action of an organ C. Nerve signal transmission is a further example of action.

The organ C is the heart of the patient P.

In one embodiment, the second device 25 may be implanted in the endocardium of the heart of the patient P, for example in the right ventricle, when the second device is in the stimulation position thereof.

The second device 25 includes a stimulator 115, a second emitter/receiver 120, and a converter 125.

The stimulator 115 is configured to stimulate the organ C. In particular, the stimulator 115 is configured to trigger a contraction of the heart of the patient P.

The stimulator 115 includes, for example, an electrode 130 connecting electrically the second device 25 to a predetermined location of the organ C of the patient P.

The second emitter/receiver 120 is configured to exchange data with the first emitter/receiver 60.

The converter 125 is configured to receive energy from the transmitter 70 and to convert the energy received into electrical energy.

In particular, the converter 125 is configured to supply the stimulator 115 with a third power supply current C3.

For example, the converter 125 is configured to receive the acoustic wave emitted by the transmitter 70 and to generate in response the third power supply current C3.

The converter 125 includes, for example, piezo-electric elements suitable for converting a force into electric voltage. In particular the piezo-electric elements are suitable for converting the acoustic wave emitted by the transmitter 70 into electric voltage.

Alternatively, the converter 125 is configured to receive an electromagnetic wave emitted by the transmitter 70 and to generate in response the third power supply current C3. The converter 125 includes, for example, a coil suitable for resonating at the frequency of the electromagnetic wave emitted by the transmitter 70.

The operation of the implantable system 10 will now be described.

During a first step prior to the implantation of the anchor 15, the centralisation device 20 and the second device 25 in the body of the patient P, the first electrical energy reserve 90 is charged with electrical energy. The first electrical energy reserve 90 therefore generates the first power supply current C1 intended for the first controller 45.

During a second step, the anchor 15, the centralisation device 20 and the second device 25 are implanted in the body of the patient P.

During a third step, an activation message is transmitted, by an external device, to the centralisation device 20. In particular, the activation message is transmitted by radiofrequency communication. The activation message informs the first controller 45 that the implantable system 10 has indeed been implanted in the body of the patient P.

During a fourth step after the third step, the first controller 45 commands the power supply of the second device 25 by the transmitter 70. For example, the first controller 45 commands the closure of a switch connecting electrically the third connector 85 to the transmitter 70. The third connector 85 then transmits the second power supply current C2 to the transmitter 70.

During the fourth step, the transmitter 70 then emits an acoustic wave and routes the acoustic wave to the second device 25.

The acoustic wave is, for example, an ultrasound wave. Ultrasound waves are acoustic waves having a frequency between 20 kilohertz and 100 megahertz.

During a fifth step, the converter 125 receives the wave emitted by the transmitter 70. The converter 125 converts at least a portion of the energy of the wave received into electrical energy. The converter 125 then generates the third electric current C3 from the wave received and supplies the stimulator 115 with the third electric current C3.

During a sixth step, the third electric current C3 is transmitted to the heart of the patient P by the electrode 130. The patient's heart then contracts in response to the third electric current C3. For example, the third electric current C3 is suitable for correcting a cardiac rhythm disorder such as a fibrillation.

During a seventh step, the first controller 45 commands the interruption of the second power supply current C2. The second device 25 is therefore no longer supplied with power, and the third electric current C3 is therefore no longer transmitted to the heart of the patient P. The stimulation of the contraction of the heart C therefore ends.

The fourth, fifth, sixth and seventh steps are, for example, repeated successively in this order with a predetermined time period. The time period is, typically, a period corresponding to the number of contractions per minute sought, for example about 70 contractions per minute.

By means of the invention, the electrical power supply of the second device 25 is provided from the centralisation device 20. The second device 25 is then a passive device, since it is only activated by the transmission of energy from the centralisation device 20. The second device 25 has the sole function of converting the wave emitted by the centralisation device 20 into an electric current transmitted to the heart C.

The second device 25 is then simple to produce. Furthermore, the volume of the second device 25 is small, since it contains no electrical energy reserve. The implantation of the second device 25 is therefore rendered easier, and is possible in a greater number of locations.

Given that the centralisation device 20 is in the stomach, the replacement of the first electrical energy reserve 90 is easy and may, for example, be carried out endoscopically via the oesophagus, simply and quickly. Furthermore, the replacement of the first electrical energy reserve 90 involves few risks of infection since no incision is made.

The use of the attractors 110 renders the positioning of the first electrical energy reserve 90 even simpler, even without endoscopy, since it is simply necessary for the patient P to swallow the first electrical energy reserve 90.

Furthermore, the implantable system 10 does not mean that the patient P continuously bears electrical energy storage means outside their body, or that unsightly electrical conductors emerge out of the body of the patient P. The implantable system 10 therefore involves few constraints for the patient.

The positioning of the centralisation device in the stomach 30 of the patient P makes it possible to interact effectively with the second device 25, for a great variety of organs C and therefore a great variety of locations of the second device 25.

Indeed, although the first example has been described in the case of cardiac stimulation, it is to be noted that the invention is suitable for being applied to a large number of separate organs C.

According to one alternative embodiment, the organ C is a nerve of the patient P.

For example, the organ C is the phrenic nerve of the patient P. For example, the second device 25 is configured to stimulate the phrenic nerve electrically.

Alternatively, the organ C is a nerve wherein the stimulation makes it possible to suppress a pain nerve signal of the patient P.

According to a further alternative embodiment, the organ C is a muscle of the patient P different to the heart. For example, the organ C is the diaphragm of the patient P.

According to a further alternative embodiment, the implantable system 10 comprises at least two second devices 25. For example, the second devices 25 are suitable, each, for stimulating a respective organ C.

Alternatively, at least two second devices 25 are configured to stimulate the same organ C. For example, one of the second devices 25 is implanted in the left ventricle and the other second device 25 is implanted in the right ventricle. The stimulation of the heart of the patient P is then closer to the physiological state than when the implantable system 10 includes a single second device 25. The implantable system 10 is then particularly suitable for the case of the treatment of certain types of heart failure.

According to a further alternative embodiment, the second device 25 does not include a second emitter/receiver 120. In this embodiment, the acoustic wave emitted by the transmitter 70 and received by the converter 125 is the only form of communication between the centralisation device 20 and the second device 25.

A second example of an implantable system 10 will now be described. Identical elements to the first example of an implantable system 10 in FIG. 1 are not described again. Only the differences are highlighted.

The stimulator 115 includes a second electrical energy reserve. The second reserve includes, for example, a capacitor. The second reserve is suitable for receiving the third power supply current C3 and for storing at least a portion of the electrical energy of the third power supply current C3.

The stimulator 115 is configured to generate an electrical pulse from the electrical energy stored in the second reserve.

The operation of the second example will now be described.

The first, second and third steps are identical to the first, second and third steps of the first example.

The fourth step has a duration strictly greater than the time period. The duration is, for example, greater than or equal to one hour, in particular greater than or equal to one week.

For example, the fourth step starts upon the reception of the activation message by the second device 25 and ends following the reception of a deactivation message. The deactivation message is a radiofrequency message. For example, the deactivation message is generated by an external device to the implantable system 10 when a medical practitioner instructs that the implantable system 10 is to be removed from the body of the patient P.

The fifth step has an identical duration to the fourth step. During the fifth step, the second reserve is supplied with the third power supply current C3. The second reserve is therefore charged progressively with electrical energy.

During the sixth step, the centralisation device 20 transmits to the emitter/receiver 120 a command message of a stimulation of the organ C by the stimulator 115.

In response to the command message, the stimulator 115 generates an electrical pulse.

The electrical pulse is conducted to the heart of the patient P by the electrode 130. The patient's heart then contracts in response to the third electric current C3.

The sixth step is implemented periodically with the time period.

The seventh step is not implemented.

In the second example, the power supply of the second device 25 is continuous. The amplitude of the wave used is then smaller. The implantable system 10 is therefore compatible with a greater number of transmitters 70.

A third example of an implantable system 10 will now be described. Identical elements to the first example of an implantable system 10 in FIG. 1 are not described again. Only the differences are highlighted.

The second device 25 includes at least one sensor 117. Each sensor 117 is suitable for measuring a value of a representative parameter of a physiological phenomenon of the patient P. The second emitter/receiver 120 is then configured to transmit to the centralisation device 20 the values measured.

The first controller 45 is configured to detect at least one physiological phenomenon occurring in the patient P. In particular, the first controller 45 is configured to detect the physiological phenomenon based on the values measured by the sensor integrated in the second device 25.

The physiological phenomenon is, for example, sleep apnoea. For example, the organ C is the phrenic nerve.

In this case, the sensor 117 is suitable for detecting a movement of the diaphragm of the patient P, indicating an inhalation. When an inhalation has not been detected for a predetermined duration, the first controller 45 detects sleep apnoea.

When the physiological phenomenon is detected, the centralisation device 20 commands in response the stimulation of the organ C. In particular, the second device 25 stimulates electrically the organ C in response to the command transmitted by the centralisation device 20.

For example, the centralisation device 20 commands the stimulation, by the second device 25, of the patient's phrenic nerve. The stimulation of the phrenic nerve then triggers a cough unblocking the upper airways of the patient P.

Alternatively, the organ C is the diaphragm. The stimulation then triggers a reflex contraction of the diaphragm which induces an inhalation.

According to a further alternative embodiment, the physiological phenomenon is a cardiac rhythm disorder, for example a bradycardia or a syncope. In this case, the sensor(s) 117 are suitable for measuring values of parameters relative to rhythm disorders such as an electrical or mechanical activity of the heart. For example, the sensor(s) 117 are suitable for measuring a difference in potential between two electrodes and/or an acceleration caused by a cardiac contraction. In this case, the second device 25 is suitable for stimulating the heart.

According to a further alternative embodiment, at least one sensor 117 is suitable for measuring a level of a biological marker in a bodily fluid F of the patient P.

According to a fourth example, the centralisation device 20 includes at least one sensor 117. For example, the centralisation device 20 includes two sensors 117.

Each sensor 117 is external to the first controller 45 but is suitable for communicating with the first controller 45.

Each sensor 117 is configured to measure values of a physiological parameter of the patient P. The physiological parameter is, for example, a parameter of the organ C.

For example, at least one sensor 117 is suitable for measuring values of a parameter of the heart.

For example, a sensor 117 is suitable for measuring a value of an acceleration of the centralisation device 20, such as an acceleration caused by a contraction of the heart C.

Alternatively or additionally, a sensor 117, for example integrated in the centralisation device 20, is suitable for measuring a value of a difference in electrical potential between two electrodes of the sensor 117. The difference in electrical potential is, for example, measured between two points of the stomach wall, i.e. the two electrodes are in contact with the stomach wall. Alternatively, the sensor 117 only includes one electrode, and is suitable for measuring the difference in electrical potential between the electrode and the anchor 15.

A fifth example of an implantable system 10 will now be described. Identical elements to the first example of an implantable system 10 are not described again. Only the differences are highlighted.

The electrical power supply 55 does not include a third connector 85 or electrical energy reserve 90.

The electrical power supply 55 includes an electrical energy generator. It is understood by “electrical energy generator” that the electrical energy generator is not configured to be charged with electrical energy by an electric current.

The electrical energy generator is suitable for generating at least one electric current by reacting at least one chemical species present in the body of the patient P. More specifically, the electrical energy generator is suitable for generating the first power supply current C1 and the second power supply current C2.

For example, the electrical energy generator comprises two electrodes, the electrodes being immersed in the gastric juices of the patient P when the centralisation device 20 is in the fixation position. Alternatively, the electrodes of the electrical energy generator are envisaged to be immersed in the intestine of the patient P when the centralisation device 20 is in the fixation position.

Each electrode includes at least one enzyme. Alternatively, each electrode includes at least one microorganism. For example, each electrode of the electrical energy generator includes an electrical conductor coated with the enzyme or microorganism, the whole thus formed being surrounded by a membrane. The membrane is, for example, configured to be traversed by certain chemical species naturally present in the stomach of the intestine of the patient P.

When the electrodes of the electrical energy generator are immersed in the gastric juices or in the intestinal fluid, one of the electrodes acts as an anode in an oxidation-reduction reaction involving a first chemical species. At the same time, the other electrode acts as a cathode in an oxidation-reduction reaction involving a second chemical species.

By the simultaneous oxidation and reduction of the first chemical species and the second chemical species, an electrical voltage appears between the two electrical conductors. The first power supply current C1 and the second power supply current C2 are then generated.

The first chemical species is, for example, glucose. The second chemical species is, for example, oxygen.

The sixth example of an implantable system 10 does not require electrical charging of an electrical energy reserve 90 or insertion of the electrical energy reserve 90 in the body of the patient P.

The constraints for the patient P are, here again, reduced.

According to a sixth example, the electrical energy generator is suitable for generating at least one electric current by converting mechanical energy into electrical energy. In particular, the electrical energy generator is suitable for generating at least one electric current from the movements of the stomach 30.

In the above description, the functions of the implantable system 10 have been separated into several examples to facilitate the comprehension thereof by the reader. However, it is to be noted that the preceding examples may be combined to give rise to new embodiments.

For example, when the second device 25 is a device for stimulating the organ C, the centralisation device 20 is suitable for including a sensor 117, the stimulation time period of the organ C then being computed by the first controller 45 based on the values measured by the sensor 117.

Furthermore, the above description has been given in the case wherein the anchor 15 and the centralisation device 20 form two separate devices. Those skilled in the art will readily understand that the centralisation device 20 and the anchor 15 are suitable for forming a single device, the anchor 15 and the centralisation device 20 then not being separable from one another. For example, the anchor 15 is integral with the housing 65 of the centralisation device 20.

According to a further example, the head 35 includes at least one base situated outside the stomach 30. For example, the head 35 includes two bases.

Each base is configured to bear against the outer face of the wall of the stomach 30 and to be connected to the implantable device 20 so as to exert a force tending to press the implantable device 20 against the inner face of the wall of the stomach 30. According to an alternative embodiment, each base is configured to be placed between the visceral layer and the parietal layer of the peritoneum and to bear against the visceral layer to press the implantable device 20 against the inner face of the wall of the stomach 30.

Each base is, for example, a plate. Alternatively, each base includes a lattice of strands stretched on a frame, in particular a flexible frame suitable for being bent and inserted into to an endoscope or a hollow needle.

The first connector 40 includes, for example, one or a plurality of rings rigidly connected to the housing 65. Each base is, for example, fixed to the implantable device 20 by one or a plurality of strands fixed to one or a plurality of rings.

Fixation by one or a plurality of bases makes it possible to distribute the pressure exerted by the implantable device over a larger surface of the wall of the stomach 30 and therefore decrease the pressure exerted. Furthermore, this fixation mode does not imply generating in the stomach wall a fold reducing the volume of the stomach, which is liable to give rise to tensions in the anchor fixed thereto. Since the forces exerted on the stomach wall are reduced, the risks of onset of an inflammatory reaction of the gastric mucosa are limited.

1. An implantable system comprising:
a first device, called centralisation device, suitable for being implanted in a fixation position inside the body of a patient and
at least one second device including an electrode suitable for electrically connecting the second device to an organ of the patient when the second device is implanted, in a stimulation position, in the body of the patient,
the first device being further configured to command the transmission, by the electrode, of an electric current for stimulating the organ by the second device,
wherein the implantable system includes an anchor including a head configured to anchor the anchor to the wall of the patient's stomach, the anchor being configured to be fixed to the first device and to hold the first device in the fixation position thereof when the anchor is fixed in the stomach, the first device being accommodated in the stomach when the first device is in the fixation position thereof.

2. The implantable system according to claim 1, wherein the first device is configured to supply the second device with power.

3. The implantable system according to claim 2, wherein the centralisation device includes an acoustic wave emitter and the second device includes a stimulator suitable for stimulating the organ and a converter of acoustic energy into electrical energy, the converter being suitable for receiving the acoustic waves emitted by the centralisation device and for generating in response an electrical power supply current of the stimulator.

4. The implantable system according to claim 3, wherein the acoustic wave is an ultrasound wave.

5. The implantable system according to claim 2, wherein the centralisation device includes an electromagnetic wave emitter and the second device includes a stimulator suitable for stimulating the organ and a converter suitable for receiving electromagnetic waves emitted by the centralisation device and for generating in response an electrical power supply current of the stimulator.

6. The implantable system according to claim 1, wherein the electrode is suitable for electrically connecting the second device to the heart of the patient.

7. The implantable system according to claim 6, including at least two second devices.

8. The implantable system according to claim 1, wherein the electrode is suitable for electrically connecting the second device (25) to a nerve of the patient (P).

9. The implantable system according to claim 8, wherein the electrode is suitable for electrically connecting the second device to the phrenic nerve of the patient (P).

10. The implantable system according to claim 1, wherein the electrode is suitable for electrically connecting the second device to the diaphragm of the patient.

11. The implantable system according to claim 1, wherein the centralisation device or the second device includes at least one sensor suitable for measuring at least one value of a parameter of the organ and the centralisation device comprises a controller suitable for commanding the stimulation of the organ, by the second device, according to the measured value(s).

12. The implantable system according to claim 1, wherein the centralisation device comprises a controller and an electrical power supply including a removable electrical energy reserve and a connector suitable for accommodating the electrical energy reserve, the electrical energy reserve being suitable for electrically powering the controller when the electrical energy reserve is connected electrically to the connector in a connection position and preferably being configured to be swallowed by the patient and to move spontaneously to the connection position from a disconnection position wherein the electrical energy reserve is accommodated in the stomach of the patient and is disconnected from the connector.

13. The implantalbe system according to claim 1, wherein the centralisation device comprises a controller and an electrical power supply suitable for generating an electrical power supply current of the controller by reacting at least one chemical species present in the body of the patient, particularly glucose.

14. The implantable system according to claim 1, wherein the centralisation device comprises a controller and an electrical power supply suitable for generating an electrical power supply current of the controller by converting mechanical energy into electrical energy.
